(19) United States
(12) Patent Application Publication
(10) Pub. No.: US 2011/0257188 A1
(43) Pub. Date: Oct. 20, 2011

(54) Compositions and methods for the treatment of nasal conditions

(76) Inventor: Gerald Horn, Deerfield, IL (US)

(21) Appl. No.: 13/066,370

(22) Filed: Apr. 13, 2011

Related U.S. Application Data
(63) Continuation-in-part of application No. 12/460,967, filed on Jul. 27, 2009.
(60) Provisional application No. 61/137,714, filed on Aug. 1, 2008.
(60) Provisional application No. 61/192,777, filed on Sep. 22, 2008.
(60) Provisional application No. 61/203,120, filed on Dec. 18, 2008.
(60) Provisional application No. 61/207,481, filed on Feb. 12, 2009.

(51) Int. Cl. A61K 31/498; A61P 27/02; A61P 11/02; A61P 27/06

(52) U.S. Cl. 514/249

(57) ABSTRACT

The invention provides compositions and methods for treating nasal congestion. The provided compositions and methods utilize low concentrations of selective α-2 adrenergic receptor agonists. The compositions preferably include brimonidine.

### CROSS-REFERENCE TO RELATED APPLICATIONS

This application is a continuation-in-part of U.S. patent application Ser. No. 12/460,967 filed Jul. 27, 2009, which claims a priority of U.S. Provisional Application Ser. Nos. 61/137,714, filed on Aug. 1, 2008; 61/192,777, filed on Sep. 22, 2008; 61/203,120, filed on Dec. 18, 2008; and 61/207,481 filed on Feb. 12, 2009. The contents of the above-mentioned applications are hereby incorporated by reference in their entirety.

### BACKGROUND OF THE INVENTION

Adrenergic receptors mediate physiological responses to the catecholamines, norephinephrine and epinephrine, and are members of the superfamily of G protein-coupled receptors having seven transmembrane domains. These receptors, which are divided pharmacologically into α-1, α-2 and β-adrenergic receptor types, are involved in diverse physiological functions including functions of the cardiovascular and central nervous systems. The α-adrenergic receptors mediate excitatory and inhibitory functions: α-1 adrenergic receptors are typically excitatory post-synaptic receptors which generally mediate responses in an effector organ, while α-2 adrenergic receptors are located postsynaptically as well as presynaptically, where they inhibit release of neurotransmitters. The α-adrenergic receptors also mediate vascular constriction. Agonists of α-2 adrenergic receptors currently are used clinically in the treatment of hypertension, glaucoma, spasticity, and attention-deficit disorder, in the suppression of opiate withdrawal, as adjuncts to general anesthesia and in the treatment of cancer pain.

α-2 adrenergic receptors are present in various bodily organs, including eyes and nose. It is believed that they play a role in nasal congestion, among many other diseases.

α-2 adrenergic receptors are presently classified into three subtypes based on their pharmacological and molecular characterization: α-2A/D (α-2A in human and α-2D in rat); α-2B; and α-2C (Bylund et al., Pharmacol. Rev. 46:121-136 (1994); and Hein and Kobilka, Neuropharmacol. 34:357-366 (1995)). The α-2A, α-2B, and α-2C subtypes appear to regulate arterial and/or venular contraction in some vascular beds, and the α-2A and α-2C subtypes mediate feedback inhibition of norepinephrine release from sympathetic nerve endings.

Many compounds having selective α-2 agonist activity are known and include brimonidine (which has been used for lowering intraocular pressure in patients with open-angle glaucoma or ocular hypertension), guanfacine (which has been used to control high blood pressure), dexmedetomidine (which has been used as a sedative, analgesic, sympatholytic and anxiolytic), and methyl dopa (which has been used as a centrally-acting adrenergic antihypertensive).

Nasal conditions, such as nasal congestion, cause inconveniences and sufferings to many patients. Thus, there is a need for new compositions and methods that would be useful for treatment of nasal conditions, including but not limited to nasal congestion.

### SUMMARY OF THE PRESENT INVENTION

The present invention provides compositions and methods for treating a nasal condition by administering low concentrations of highly selective α-2 adrenergic receptor agonists to a patient in need thereof.

The provided compositions and methods utilize low concentrations of highly selective α-2 adrenergic receptor agonists having a binding affinity of 100 fold or greater for α-2 over α-1 adrenergic receptors. The concentration of the selective α-2 adrenergic receptor agonist is preferably below the concentration at which α-1 adrenergic receptors are activated sufficiently enough to cause adverse ischemic vasoconstrictive consequences. Preferably, the concentration of the selective α-2 adrenergic receptor agonist is below about 0.05% weight by volume of the composition.

In preferred embodiments of the invention, the selective α-2 adrenergic receptor agonist is selected from the group consisting of apraclonidine, mivazerol, clonidine, brimonidine, alpha methyl dopa, guanfacine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

In a preferred embodiment, a pH of the composition comprising the selective α-2 adrenergic receptor agonist is between about 4.0 and about 8.5. If it is desired to achieve a more effective topical mucosal application with minimal mucosal penetration (for example, in such conditions as vasomotor rhinitis or nasal congestion), then it is generally preferred to maintain pH of the composition between about 4.0 and about 6.5. If, on the other hand, it is desired to achieve a deeper mucosal penetration (for example, in such conditions as allergic rhinitis or sleep apnea), then a preferred pH of the composition is between about 6.5 and about 8.0.

In one embodiment of the invention, the compositions of the invention can be administered by nasal delivery. In another embodiment of the invention, the compositions of the invention can be administered by topical ophthalmic delivery.

### BRIEF DESCRIPTION OF THE FIGURES

FIG. 1 is a graphical representation of the effects of activating α-1 adrenergic receptors; and

FIG. 2 is a graphical representation of the effects of preferentially activating α-2 adrenergic receptors.

### DETAILED DESCRIPTION OF THE INVENTION

### Definitions

For purposes of the present invention, the terms below are defined as follows.

The term “low concentrations” refers to concentrations from between about 0.0001% to about 0.05%; more preferably, from about 0.001% to about 0.025%; even more preferably, from about 0.01% to about 0.025%; and even more preferably, from about 0.01% to about 0.02% weight by volume of the composition.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan™, and UK14304.

The terms “treating” and “treatment” refer to reversing, alleviating, inhibiting, or slowing the progress of the disease, disorder, or condition to which such terms apply, or one or more symptoms of such disease, disorder, or condition.

The term “nasal condition” refers to any disease, disorder, or condition which affects and/or involves the nose. This term includes, but is not limited to, such conditions as nasal congestion, diseases and/or conditions associated with swollen nasal turbinates, all types of rhinitis including but not limited to vasomotor rhinitis and allergic rhinitis, sleep apnea, acute or chronic sinusitis, nasal polyposis, and any disease and/or condition associated with nasal discharge.

The term “substantial enlargement of nasal turbinates” refers to a significant enlargement of nasal turbinates, for example, more than about 50% compared to the baseline level of the patient so that it negatively affects the patient's breathing.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

### Embodiments of the Invention

It was surprisingly and unexpectedly found that selective alpha-2 (α-2) adrenergic receptor agonists (which are interchangeably referred to as “α-2 agonists” throughout the application) with extremely high selectivity for α-2 adrenergic receptors at sufficiently low concentrations and at pH of between about 4.0 and about 8.5 can be used to treat a nasal condition in a patient in need thereof.

One example of a nasal condition that may be treated with the compositions and methods of the present invention is nasal congestion. Nasal congestion is turbinate mucosal swelling which is caused by, or is contributed by, vasodilation of blood vessels. While not wishing to be bound to any particular theory, it is believed that vasodilation is primarily associated with α-1 adrenergic receptors activity. Thus, unless the binding affinity of α-2 agonists for α-2 over α-1 adrenergic receptors is sufficiently high, insufficiently highly selective α-2 agonists cause undesirable α-1 receptor stimulation with attendant vasodilation.

Accordingly, the invention is directed to compositions and methods which employ highly selective α-2 agonists that have minimal α-1 agonist activity. The compositions of the present invention preferentially stimulate α-2 adrenergic receptors so that α-1 adrenergic receptors are not stimulated sufficiently enough to cause vasodilation.

Thus, in one embodiment, the invention provides a method of treating a nasal condition comprising administering to a patient in need thereof a selective α-2 adrenergic receptor agonist having a binding affinity of 100 fold or greater for α-2 over α-1 adrenergic receptors, or a pharmaceutically acceptable salt thereof, wherein said selective α-2 adrenergic receptor agonist is present at a concentration below about 0.05% weight by volume.

In one embodiment, the invention provides compositions formulated for treating a condition associated with swollen nasal turbinates. Compositions particularly useful for these purposes preferably comprise brimonidine at concentrations of from 0.01% to about 0.04%, and more preferably, from 0.02% to about 0.035%. In a preferred embodiment, the condition associated with swollen nasal turbinates is selected from the group consisting of nasal congestion, allergic rhinitis, asthma, sleep disorders, and sleep apnea. A preferred pH of the composition formulated for the condition associated with swollen nasal turbinates is between about 6.5 and about 8.5.

### Selective α-2 Adrenergic Receptor Agonists Suitable for the Purposes of the Invention

Selective α-2 agonists that may be used for the purposes of the present invention have extremely high selectivity for α-2 adrenergic receptors, defined by their binding affinities (Kᵢ) for α-2 over α-1 receptors of more than 100:1, more preferably 300:1; more preferably 500:1, even more preferably 700:1, even more preferably 1000:1 or greater, and most preferably, 1500:1 or greater.

Not desiring to be bound by any specific theory or mechanism, it is believed that the particularly preferred adrenergic receptor agonists for the purposes of the present invention are highly selective for α-2B and/or α-2C receptors, as opposed to α-2A receptors.

In one embodiment, the selective α-2 adrenergic receptor agonist is a compound which has binding affinity of about 100 fold or greater for α-2 over α-1 adrenergic receptors, preferably about 500 fold or greater, more preferably about 700 fold or greater, even more preferably about 1000 fold or greater, and most preferably, about 1500 fold or greater.

The selective α-2 adrenergic receptor agonist may be present at a concentration from between about 0.0001% to about 0.05%; more preferably, from about 0.001% to about 0.025%; even more preferably, from about 0.01% to about 0.025%; and even more preferably, from about 0.01% to about 0.02% weight by volume.

It is preferred that a concentration of a selective α-2 adrenergic receptor agonist be below its vasoconstriction vs. concentration plateau. Typically, the optimal concentration is 10% to 90% above the minimal threshold of measurable vasoconstriction for a particular α-2 agonist, or below that of the plateau maximum concentration, and is preferably within the about 25% to about 75% range of either of these benchmarks. The term “plateau maximum concentration” means the concentration above which there is no or minimal further vasoconstriction effect. Other considerations in choosing a selective α-2 adrenergic receptor agonist are blood brain permeability and any possible side effects and other systemic reactions.

In one embodiment, the selective α-2 adrenergic receptor is selected from the group consisting of apraclonidine, mivazerol, clonidine, brimonidine, alpha methyl dopa, guanfacine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds. Analogs of these compounds that function as highly selective α-2 agonists may also be used in compositions and methods of the present invention.

In a more preferred embodiment, the selective α-2 adrenergic receptor is brimonidine in the form of a salt. In a preferred embodiment, the salt is tartrate salt.

### Compositions and Methods of the Invention

In one embodiment, the invention provides a composition comprising a selective α-2 adrenergic receptor agonist having a binding affinity of 100 fold or greater for α-2 over α-1 adrenergic receptors, or a pharmaceutically acceptable salt thereof, for treating nasal congestion.

In a preferred embodiment, said selective α-2 adrenergic receptor agonist is present at a concentration below about 0.05% weight by volume, and more preferably, between about 0.001% to about 0.05% weight by volume.

In one embodiment, the selective α-2 adrenergic receptor agonist is selected from the group consisting of lofexidine, apraclonidine, mivazerol, clonidine, brimonidine, alpha methyl dopa, guanfacine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

In a preferred embodiment, the composition comprises brimonidine at a concentration between about 0.001% and about 0.025% weight by volume.

In a preferred embodiment, a pH of the composition comprising the selective α-2 adrenergic receptor agonist is between about 4.0 and about 8.5.

If it is desired to achieve a more effective topical mucosal application with minimal mucosal penetration (for example, in such conditions as vasomotor rhinitis or nasal congestion with substantial nasal discharge but relatively minimal turbinate swelling or physical blockage of nasal passages), then it is generally preferred to maintain pH of the composition between about 4.0 and about 6.5.

There is a direct relationship between a selective α-2 agonist's lipophilicity (as characterized by the Log D value) and the pH of a pharmaceutical composition containing the selective α-2 agonist: as the pH increases across the range of 4.0 to 8.5, the selective α-2 agonist's lipophilicity exponentially increases. This correlation is true for virtually all selective α-2 agonists, and in particular, brimonidine and dexmedetomidine.

Log D refers to a lipophilicity value at a given pH. This measurement is especially useful to determine the level of topical lipophilicity and resultant permeability of a topical composition. The higher the lipophilicity, the greater is the selective α-2 agonist's penetration through the lipophilic mucosal epithelial cell membranes. This is because at a more alkaline pH, more of the compound is present in a non-ionized form. When the pH is relatively low, e.g. between about 4.0 and about 6.5, the selective α-2 agonist is relatively less lipophilic and more ionized. As a result, a greater percentage of the selective α-2 agonist remains on the mucosa, increasing the drug's effectiveness as compared to the results at a higher pH. Thus, pH range of 4.0 to about 6.5, and more preferably 4.0 to 5.8, is preferred for the formulations for the treatment of nasal conditions involving serous nasal discharge without substantial turbinate swelling, such as vasomotor rhinitis.

If, on the other hand, it is desired to achieve a deeper mucosal penetration (for example, in such conditions as allergic rhinitis or sleep apnea; and generally in any nasal condition involving substantial enlargement of the nasal turbinates and/or physical blockage of nasal passages), then a preferred pH of the composition is between about 6.5 and about 8.5. At this higher pH, a greater proportion of the α-2 agonist will be non-ionized and more lipophilic, resulting in the greater permeation of the α-2 agonist through the lipophilic mucosal epithelial cell membranes. Thus, pH range of 6.5 to 8.5, and more preferably, 7.5 to 8.5 is preferred for formulations for the treatment of allergic rhinitis, sleep apnea, and other disorders associated with substantial enlargement of nasal turbinates and/or physical blockage of nasal passages, for example due to venous sinusoidal dilation.

For some nasal conditions, it may be preferred to achieve a moderate lipophilicity, which is associated with pH of between about 5.6 and 6.2.

Dexmedetomidine has the following Log D values at different pH:

pH 4.0 to 5.6: Log D is 0.76 to 1.76;

pH 5.6 to 6.2: Log D is 1.76 to 2.28;

pH 6.2 to 8.0: Log D is 2.28 to 3.00.

The lower the Log D value is, the less is lipophilicity and the more is surface retention and mucosal effectiveness. Conversely, the higher the Log D value is, the more is lipophilicity, and the more is mucosal penetration and submucosal permeation.

Brimonidine has the following Log D values at different pH:

pH 4.0 to 6.2: Log D is -1.02 to -0.44;

pH 7.0 to 8.0: Log D is 0.55 to 0.79.

When the selective α-2 agonist is brimonidine, the moderate lipophilicity is achieved at pH of between 6.2 and 6.8. A pH of less than 6.2 is preferred to achieve greater mucosal surface retention, and a pH of greater than 6.8 is preferred to achieve greater mucosal penetration and submucosal permeation.

In one embodiment, the invention provides an aqueous composition for treating a nasal condition consisting essentially of brimonidine, wherein said brimonidine concentration is from between about 0.01% to about 0.02% weight by volume, wherein pH of said composition is between about 6.2 and about 6.8.

In one embodiment, the invention provides an aqueous composition for treating nasal congestion comprising brimonidine and from between about 0.1 to about 0.5% weight by volume of potassium chloride, wherein said brimonidine concentration is from between about 0.01% to about 0.025% weight by volume, wherein pH of said composition is between about 6.2 and about 6.8.

The compositions of the present invention are preferably formulated for a mammal, and more preferably, for a human.

In another preferred embodiment, the compositions of the present invention further include potassium (i.e., K⁺). The term “potassium” includes, but is not limited to, potassium salt. In a preferred embodiment, potassium is in the form of potassium chloride and its concentration is between about 0.2% to about 0.9% weight by volume.

In another preferred embodiment, the compositions of the present invention further include calcium (i.e., Ca²⁺). The term “calcium” includes, but is not limited to, calcium salt. Preferably, calcium is calcium chloride.

In a more preferred embodiment the compositions of the present invention contain the electrolyte KCL in a concentration range of 0.1% -0.8% weight by volume, preferably 0.25% weight by volume for a more prolonged duration of action.

In another preferred embodiment, the compositions of the invention also comprise a solubility stabilizer which preferably contains an anionic component, such as peroxide class preservatives. The solubility stabilizer allows one to achieve greater penetration of lipophilic membranes, such as those present at the vascular endothelial surface. In a preferred embodiment, the solubility stabilizer comprises a stabilized oxychloro complex, chlorite, and sodium perborate.

In yet another preferred embodiment, the compositions of the present invention comprise nitrous oxide inhibitors. In a preferred embodiment, the nitrous oxide inhibitors are selected from the group consisting of L-NAME (L-N^{G}-Nitroarginine methyl ester), L-NIL (N6-(1-Iminoethyl)-L-lysine dihydrochloride), L-NIO (N5-(1-Iminoethyl)-L-ornithine dihydrochloride), and L-canavine, or combinations thereof. Preferably, concentration of the nitrous oxide inhibitors is between about 0.005% and about 0.5% weight by volume.

The compositions of the invention may also include additional components, which include, but are not limited to, preservatives, delivery vehicles, tonicity adjustors, buffers, pH adjustors, antioxidants, and water.

The preservatives include, but are not limited to, benzalkonium chloride, chlorobutanol, thimerosal, phenylmercuric acetate, or phenylmercuric nitrate. Vehicles useful in a topical composition include, but are not limited to, polyvinyl alcohol, povidone, hydroxypropyl methyl cellulose, poloxamers, carboxymethyl cellulose, hydroxyethyl cellulose and purified water. It is also possible to use a physiological saline solution as a major vehicle.

A tonicity adjustor also can be included, if desired, in a topical composition of the invention. Such a tonicity adjustor can be, without limitation, a salt such as sodium chloride, potassium chloride, mannitol or glycerin, or another pharmaceutically or ophthalmically acceptable tonicity adjustor.

Various buffers and means for adjusting pH can be used to prepare topical compositions of the invention. Such buffers include, but are not limited to, acetate buffers, citrate buffers, phosphate buffers and borate buffers. It is understood that acids or bases can be used to adjust the pH of the composition as needed. Topically acceptable antioxidants useful in preparing a topical composition include, yet are not limited to, sodium metabisulfite, sodium thiosulfate, acetylcysteine, butylated hydroxyanisole and butylated hydroxytoluene.

The compositions of the invention may be administered through nasal delivery or delivered as ophthalmic solutions into the eyes.

In one embodiment, the provided composition is an aerosolized composition. It is within a skill in the art to prepare aerosolized compositions of the present invention. The aerosolized compositions of the present invention are generally delivered via an inhaler, jet nebulizer, or ultrasonic nebulizer which is able to produce aerosol particles with size of between about 1 and 10 μm.

To make the topical compositions of the present invention, one can simply dilute, using methods known in the art, more concentrated solutions of selective α-2 agonists. The precise method of carrying out the dilutions is not critical. Any commonly used diluents, including preservatives described above in the application, suitable for topical solutions can be used.

Proper dosages of the compositions of the present invention are concentration-dependent. To determine the specific dose for a particular patient, a skilled artisan would have to take into account kinetics and absorption characteristics of the particular highly selective α-2 adrenergic receptor agonist.

The present invention is more fully demonstrated by reference to the accompanying drawings.

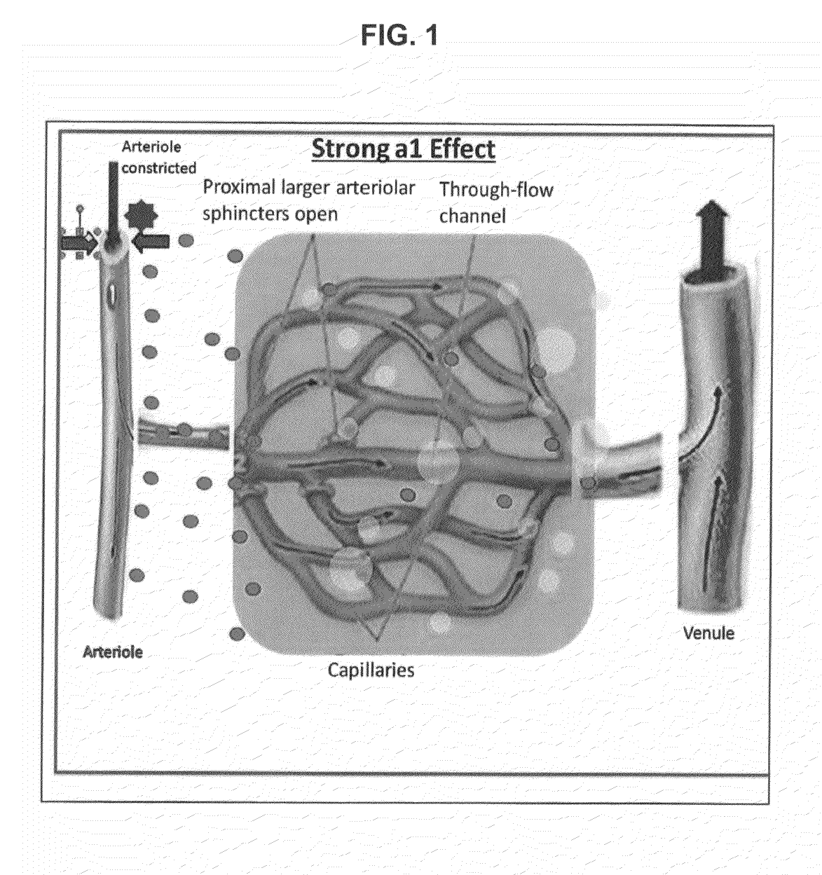

FIG. 1 is a graphical representation of the effects of activating α-1 adrenergic receptors. As FIG. 1 demonstrates, administering α-1 adrenergic receptor agonists leads to constriction of the proximal arteriole (on the left side) which in turn decreases the flow of blood through the capillaries and causes ischemia for the tissues downstream of arteriole.

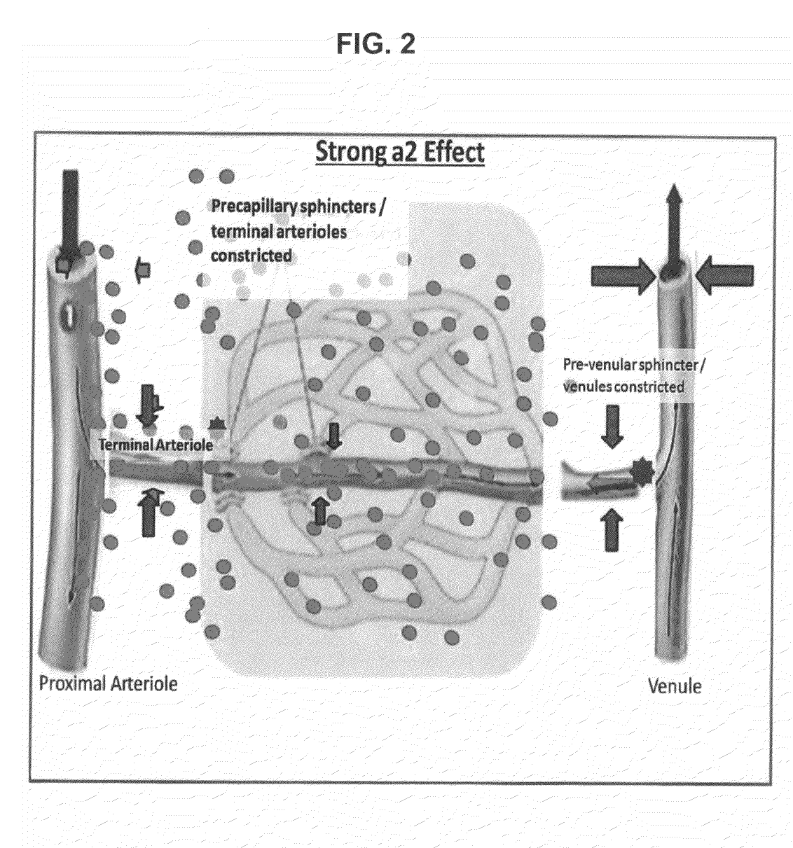

FIG. 2 is a graphical representation of the effects of preferentially activating α-2 adrenergic receptors. As FIG. 2 demonstrates, administering α-2 adrenergic receptor agonists leads to constriction of the pre-capillary/terminal arteriole (on the left side) and constriction of the venule (on the right side). Ischemia is decreased, as compared to stimulating α-1 adrenergic receptors because the arteriole is open and some oxygen is available to surrounding tissues by means of the through-flow vessels that connect the arterioles and the venules. Pre-venule constriction may reduce the ischemic effect and reduce vasodilation that may contribute to nasal congestion.

The following Examples are provided solely for illustrative purposes and are not meant to limit the invention in any way.

### EXAMPLES

### Example 1

### Ipsilateral Ophthalmic/Nasal Effectiveness Test

11 individuals were asked to assess the patency of each nostril by alternately closing one. They were then given 0.025% brimonidine topically to one randomly selected eye. After 10 minutes each nostril was alternately closed to assess the patency of the contralateral nostril and compare to its patency before applying brimonidine to the eye. 9 of the 11 patients experienced a noticeable increase in patency in the ipsilateral (i.e., on the same side as the eye) nostril, but not in the contralateral (on the opposite side of the eye) nostril. Nasal patency refers to a basic evaluation of the degree to which a nostril is open (i.e. unblocked).

Thus, this example demonstrates that ophthalmic delivery of nasal decongestants can be used to achieve significant drug concentrations in nasal turbinates, as drug flows through the nasolacrimal duct into the nasal turbinates.

### Example 2

### Effect of Brimonidine on Increasing Whiteness of an Eye and Nasal Decongestion

Eight (8) human subjects were administered 0.025% brimonidine. The subjects were administered with the drug in one eye and then asked to assess themselves in the mirror to see if they perceived a difference in conjunctival hyperemia between eyes. The drug was administered around 9:15 am. The assessments were made 5 minutes after the administration and 4 hours after the administration. After the four hours assessment, the drug was re-administered.

The results of the experiment are as follows. At the initial 5 min assessment, eight of eight subjects reported reduced hyperemia and increased whiteness in the eye to which brimonidine was administered. At the four hour assessment, eight of eight subjects reported reduced hyperemia and increased whiteness in the eye to which brimonidine was administered. Also, at the four hour assessment, six of eight subjects reported reduced nasal congestion in the nostril on the same side as the eye into which the drug was administered.

As this Example demonstrates, in several subjects, administration of brimonidine into the eye resulted in reducing nasal congestion in the nostril on the same side as the eye into which the drug was administered.

### Example 3

### Effect of Brimonidine on Increasing Whiteness of an Eye and Nasal Decongestion

Five (5) human subjects which stated that they had no previous nasal breathing problems took part in the experiment, of whom three human subjects returned the records.

1 drop of 0.025% brimonidine was applied to the right eye of each patient. In all patients, the right eye has become whiter. Then, the breathing function was measured in each nostril separately 10 minutes later. Then, 1 dose of 0.0045% brimonidine nasal spray was applied into the left nostril and the ease of breathing was again measured in each nostril separately 10 minutes later.

Following the administration of 0.025% brimonidine to the right eye, all three patients reported reducing nasal congestion in the right nostril. Following the administration of 0.0045% brimonidine nasal spray into the left nostril, all three patients reported reduced nasal congestion in the both nostrils. As this Example demonstrates, administration of brimonidine into the eye resulted in reducing nasal congestion in the nostril on the same side as the eye into which the drug was administered.

What is claimed is:

1. A composition comprising a selective α-2 adrenergic receptor agonist having a binding affinity of 300 fold or greater for α-2 over α-1 adrenergic receptors, or a pharmaceutically acceptable salt thereof, wherein pH of said composition is between about 4.0 and about 8.5.

2. The composition of claim 1, wherein said selective α-2 adrenergic receptor agonist is selected from the group consisting of lofexidine, apraclonidine, mivazerol, clonidine, brimonidine, alpha methyl dopa, guanfacine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

3. The composition of claim 1, wherein said α-2 adrenergic receptor agonist is present at a concentration from between about 0.001% to about 0.05% weight by volume.

4. The composition of claim 1, wherein said α-2 adrenergic receptor agonist is brimonidine at a concentration from between about 0.01% to about 0.025% weight by volume.

5. The composition of claim 1, further comprising from between about 0.1 to about 0.5% weight by volume of potassium chloride, and wherein said α-2 adrenergic receptor agonist is brimonidine, wherein said brimonidine concentration is from between about 0.01% to about 0.025% weight by volume, and wherein pH of said composition is between about 5.0 and about 8.0.

6. A method of treating a nasal condition in a patient in need thereof comprising administering to said patient an pharmaceutically effective amount of the composition of claim 1.

7. The method of claim 6, wherein said nasal condition is associated with severe nasal discharge with minimal turbinate swelling.

8. The method of claim 7, wherein said nasal condition comprises vasomotor rhinitis.

9. The method of claim 6, wherein said nasal condition is associated with substantial enlargement of nasal turbinates and/or physical blockage of nasal passages.

10. The method claim 9, wherein said nasal condition comprises a condition selected from the group consisting of allergic rhinitis, viral rhinitis, and sleep apnea.

11. The method of claim 9, wherein said selective α-2 adrenergic receptor agonist is brimonidine at sufficient lipophilicity to achieve substantial mucosal penetration and submucosal permeation.
